(19) United States
(12) Patent Application Publication
(10) Pub. No.: US 2022/0301698 A1
(43) Pub. Date: Sep. 22, 2022

(54) MANAGEMENT SYSTEM FOR MANAGING RECORDS REGARDING STERIALIZATION PROCESS, INFORMATION PROCESSING APPARATUS, COMPUTER-READABLE STORAGE MEDIUM, AND MANAGEMENT METHOD

(71) Applicant: CANON KABUSHIKI KAISHA, Tokyo (JP)

(72) Inventor: Motohiro Furusawa, Shizuoka (JP)

(21) Appl. No.: 17/617,662

(22) PCT Filed: Jun. 24, 2020
(86) PCT No.: PCT/JP2020/024700

§ 371 Date: Dec. 9, 2021

(30) Foreign Application Priority Data
Jul. 16, 2019 (JP) 2019-131398

(51) Int. Cl. G16H 40/20; G16H 40/40

(52) U.S. Cl. CPC G16H 40/20; G16H 40/40

(57) ABSTRACT

There is provided a management system that manages records with respect to sterilization targets on which sterilization process is to be performed. The management system includes: recording means for recording, when a first sterilization process is again performed on a first sterilization target with out the first sterilization target having been used, re-sterilization related information to a database in association with a target identifier for identifying the first sterilization target or a set including the first sterilization target; and generating means for generating statistical information regarding re-sterilization based on re-sterilization related information recorded in the past. The re-sterilization related information includes information indicating a cause of the re-sterilization.

### TECHNICAL FIELD

The present invention relates to a management system for managing records regarding sterilization process, an information processing apparatus, a computer-readable storage medium, and a management method.

### BACKGROUND ART

In hospitals, sterilizing processes are performed on sterilization targets such as medical instruments. On this occasion, so-called sterilization indicators such as a chemical indicator(hereinafter, CI) and a biological indicator(hereinafter, BI) are used in order to determine the achievement degree of the sterilization process. The CI includes a discoloration area whose color changes in accordance with the achievement degree as to a condition required for the sterilization process in which sterilizing agents (such as vapor, hydrogen peroxide, or ethylene oxide) are used. The achievement degree of the sterilization process can be determined based on the color of the discoloration area of a CI after the sterilization process. The BI includes indicator bacteria that are highly resistive to the sterilizing agent, and the indicator bacteria die out in accordance with the achievement degree as to a condition required for the sterilization process. By cultivating the BI and checking if the indicator bacteria exists after the sterilization process, the achievement degree of the sterilization process can be determined. In addition to the CI and BI, an operation log (data in which temperature and pressure at the time of operation are recorded along with time, for example) output from a sterilization processing apparatus may be used, as a physical indicator, to verify whether or not the sterilization process is successful. Japanese Patent No. 3414291 discloses a management apparatus that manages records of sterilization processes. According to Japanese Patent No. 3414291, a date and time of a process of sterilizing a sterilization target, a sterilization condition, and a CI determination result are recorded.

### SUMMARY OF INVENTION

The sterilized medical instrument is stored in a dedicated storage cabinet, and is used again on a later medical occasion. On the other hand, an instrument whose predetermined storage period has expired, or an instrument having a risk of bacteria being attached thereon due to being dropped or the like when a work such as delivery from the storage cabinet or opening is performed is again subjected to the sterilization process without being used. However, in general, resources for sterilization (such as apparatuses for cleaning and sterilization, work spaces for assembly and packaging, human resources, and the like) that can be used in an individual hospital are limited. Therefore, it is desirable to avoid performing re-sterilization (sterilization being performed again without using it) on a medical instrument that originally need not be sterilized, and to appropriately use the hospital's limited resources, from the viewpoints of safety and productivity improvement in medical activities.

This disclosure is to provide a mechanism for assisting appropriate utilization of hospital resources regarding sterilization.

According to a first aspect, there is provided a management system that manages records with respect to sterilization targets on which sterilization process is to be performed. The management system includes: recording means for recording, when a first sterilization process is again performed on a first sterilization target with out the first sterilization target having been used, re-sterilization related information to a database in association with a target identifier for identifying the first sterilization target or a set including the first sterilization target; and generating means for generating statistical information regarding re-sterilization based on re-sterilization related information recorded in the past. The re-sterilization related information includes information indicating a cause of the re-sterilization.

According to a second aspect, there is provided a management system that manages records with respect to sterilization targets that are stored based on event-related sterility maintenance. The management system includes: recording means for recording a sterilization processing date on which a sterilization process has been performed with respect to at least one set of a sterilization target to a database; and searching means for searching the database based on a storage period, before an occurrence of an event, of a first sterilization target that relates to the occurred event to specify a sterilization target, out of other stored sterilization targets, that should be subjected to re-sterilization.

Further features of the present invention will become apparent from the following description of exemplary embodiments with reference to the attached drawings.

### BRIEF DESCRIPTION OF DRAWINGS

FIG. 1 is a diagram illustrating a sterilization workflow according to an embodiment.

FIG. 2 is a diagram illustrating a configuration of a management system according to an embodiment.

FIG. 3 is a diagram illustrating a CI according to an embodiment.

FIG. 4 is a diagram illustrating a configuration of a measurement apparatus according to an embodiment.

FIG. 5 is a diagram illustrating a first example of a configuration of sterilization related information according to an embodiment.

FIG. 6 is a diagram illustrating a second example of the configuration of the sterilization related information according to an embodiment.

FIG. 7 is a diagram illustrating a third example of the configuration of the sterilization related information according to an embodiment.

FIG. 8 is a diagram illustrating a fourth example of the configuration of the sterilization related information according to an embodiment.

FIG. 9 is a diagram illustrating a fifth example of the configuration of the sterilization related information according to an embodiment.

FIG. 10 is a flowchart illustrating a first example of a processing flow according to an embodiment.

FIG. 11 is a diagram illustrating a first example of statistical information according to an embodiment.

FIG. 12 is a diagram illustrating a second example of the statistical information according to an embodiment.

FIG. 13 is a diagram illustrating a third example of the statistical information according to an embodiment.

FIG. 14 is a block diagram illustrating an example of a functional configuration of the management system according to an embodiment.

FIG. 15 is a diagram illustrating an example of a configuration of ERSM management information according to an embodiment.

FIG. 16 is a diagram illustrating a proposal of re-sterilization based on the ERSM management information according to an embodiment.

FIG. 17 is a flowchart illustrating a second example of a processing flow according to an embodiment.

FIG. 18 is a diagram illustrating a first example of a screen for presenting an event occurrence status according to an embodiment.

FIG. 19 is a diagram illustrating a second example of the screen for presenting the event occurrence status according to an embodiment.

FIG. 20 is a block diagram illustrating another example of the functional configuration of the management system according to an embodiment.

### DESCRIPTION OF EMBODIMENTS

Hereinafter, embodiments will be described in detail with reference to the attached drawings. Note, the following embodiments are not intended to limit the scope of the claimed invention. Multiple features are described in the embodiments, but limitation is not made an invention that requires all such features, and multiple such features may be combined as appropriate. Furthermore, in the attached drawings, the same reference numerals are given to the same or similar configurations, and redundant description thereof is omitted.

### First Embodiment

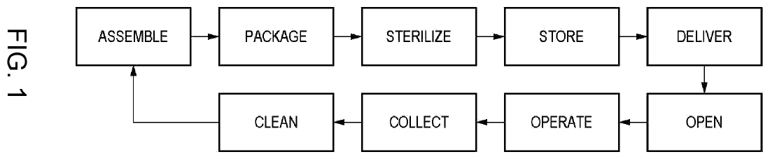

FIG. 1 shows a sterilization workflow according to the present embodiment. In the present embodiment, the sterilization workflow means a series of process stages that are to be performed regarding sterilization of a sterilization target. An assembly stage is a process stage in which one or more sterilization targets are grouped in a sterilization station. Note that the number of sterilization targets included in one group may be one. The grouping is performed based on a correspondence relationship between operations in which the sterilization targets are to be used and sterilization processing apparatuses to be used in the sterilization process of the sterilization targets. For example, sterilization targets that are to be used in the same operation, and on which the sterilization process are performed by the same sterilization processing apparatus may be grouped into a same group. A packaging stage is a process stage in which one set is formed by packaging, in a sterilization station, one or more sterilization targets of one group and a CI using one packaging member. Usually, in hospitals or the like, a plurality of sterilization targets of the same type are used, and therefore a plurality of sets each including the same sterilization target are formed. Hereinafter, each set including the same sterilization target is referred to as a set of the same type. Conversely, types of two sets being different means that at least one of sterilization targets included in the respective two sets is different. A sterilization stage is a process stage in which the sterilization process is performed on a packaged set using a sterilization processing apparatus.

A storing stage is a process stage in which a set on which the sterilization process has been performed in the sterilization stage is moved to and stored in a storage cabinet. A delivery stage is a process stage of moving a set to be used in an operation or the like from the storage cabinet to a place of usage such as an operation room. An opening stage is a process stage of opening the package of a set that has been moved to the place of usage. An operation stage is also a utilizing (using) stage in which the sterilization target is utilized or used. Note that the achievement degree of the sterilization process is determined using the CI that is enclosed in the set in the opening stage, the operation stage, or both of the stages. Then, only when the achievement degree of the sterilization process is successfully determined to be sufficiently high using the CI, the sterilization target is actually used in the operation stage. Immediately after the sterilization stage as well, it may be determined whether or not the sterilization process is successful using a CI or a physical indicator. Note that there is no limitation to these examples, and it may be determined whether or not the sterilization target can be actually used at any point in time in a period from the end of the sterilization process until the start of using the sterilization target in an operation or the like. A collection stage is a process stage in which, after a sterilization target is used, the sterilization target is moved to the sterilization station. A cleaning stage is a process stage in which a sterilization target is cleaned in the sterilization station. After a set is cleaned in the cleaning stage, the set is again transferred to the assembly stage.

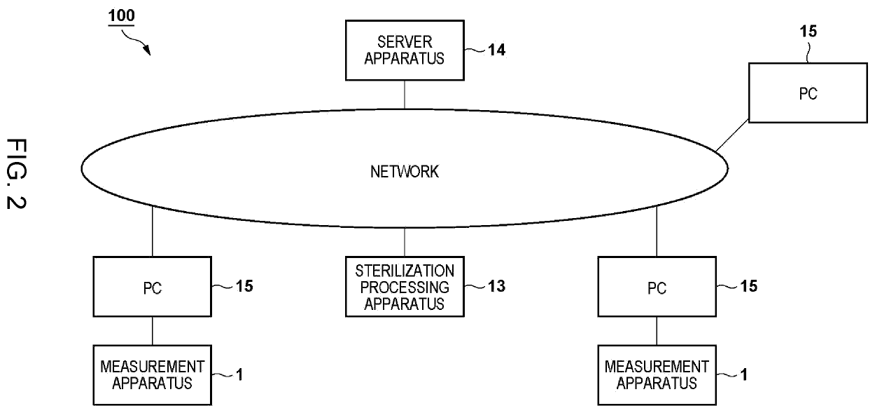

FIG. 2 is a diagram illustrating a configuration of a management system 100 that manages records regarding sterilization targets on which the sterilization process is performed. A server apparatus 14, a plurality of personal computers (hereinafter, PCs) 15, and a sterilization processing apparatus 13 are connected to a network. The server apparatus 14, the PCs 15, and the sterilization processing apparatus 13 can communicate to each other via the network. The server apparatus 14 manages sterilization related information regarding the sterilization process. The PCs 15 can add information to the sterilization related information retained by the server apparatus 14, modify the retained information, and the like via the network. Also, the PCs 15 and the server apparatus 14 can present various types of information to a user of the management system 100 based on the sterilization related information retained by the server apparatus 14. The presentation of various types of information are performed through any method such as displaying presentation contents in a display of a PC 15, outputting presentation contents to a printer (not shown) connected to a network, or outputting/transmitting information indicating presentation contents to a PC (not shown) that a user uses. As described above, although there are various methods regarding the manner with which various types of information are presented/displayed to a user, these are collectively referred to as “the management system 100 presenting/displaying (information to a user)” in the following. Note that, in the present invention, the user means a person related to at least sterilization such as operators that actually perform works in the respective process stages in the sterilization workflow, a supervisor that supervises the operators, and a manager trying to optimize utilization of resources in a hospital.

The PCs 15 are installed at places where, in the sterilization workflow, sterilization related information needs to be referred to, information needs to be added to the sterilization related information, and the sterilization related information needs to be corrected. Also, as shown in FIG. 1, some PCs 15 are connected to respective measurement apparatuses 1. Each measurement apparatus 1 is an apparatus that measures a discoloration area of a Cl. The measurement apparatuses 1 may be directly connected to the network instead of being connected to a PC 15. Also, the measurement apparatuses 1 may include a display for displaying a Cl result. Note that the network in FIG. 2 is not limited to a network that is closed inside a hospital. For example, the sterilization station may be located outside a hospital, and in this case, the network connects the hospital and the sterilization station outside the hospital. Also, the network may be a closed network such as a local area network (LAN) that is not connected to an other network, or a network including the LAN and the Internet. For example, the network can be constituted by the Internet and a LAN inside a hospital to be connected to the Internet, and in this case, the management system 100 can be configured by installing the server apparatus 14 outside the hospital and connecting it to the Internet.

Also, in the present embodiment, the sterilization processing apparatus 13 is connected to the network, and the PCs 15 and the server apparatus 14 can acquire information to be added to the sterilization related information and information for modifying the sterilization related information from the sterilization processing apparatus 13. However, the configuration may also be such that the sterilization processing apparatus 13 is not connected to the network. In this case, an operator performs addition, modification, or the like of the sterilization related information by operating a PC 15 based on information output from the sterilization processing apparatus 13. Note that, in order for an operator to perform input, update, display, or the like of the sterilization related information, the operator needs to operate a PC 15 or the like, but the description of operating a PC 15 will be omitted in the following in order to simplify the description. For example, an operator inputting information to the management system 100 by operating the PC 15 is also represented as the operator inputting information to the management system 100.

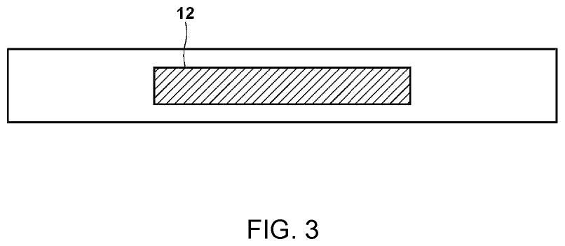

FIG. 3 shows a CI according to the present embodiment. The CI is a sheet-like test paper and includes a discoloration area 12 in which a chemical treatment is performed on its surface. The discoloration area 12 changes its color according to the achievement degree of a sterilization process. In the present embodiment, the discoloration area 12 of a CI is assumed to be configured such that its color value is a color value C#1 in an initial state, changes to a color value C#2, . . . , and to a color value C#9 according to the achievement degree of the sterilization process, and finally stabilizes at a color value C#10. Hereinafter, the achievement degree of the sterilization process at which a color value C#k (k is an integer from 1 to 10) is obtained is defined as an achievement degree #k. Note that the discoloration area 12 does not take one of the color values C#1 to C#10, and continuously change its color from the color value C#1 to the color value C#10. That is, the discoloration area 12 can take a color between a color value C#m (m is an integer from 1 to 9) and a color value C#m+1. In the present embodiment, the level of the achievement degree of the sterilization process is evaluated in nine stages. Specifically, if the color of the discoloration area 12 is in a range between a color value C#m and a color value C#m+1, the level of the sterilization process is determined to be a level #m. Also, it is assumed that levels #4 to #9 are OK levels and levels #1 to #3 are NG levels, for example. Here, the level of the sterilization process being a level #m means that the achievement degree of the sterilization process is in a range from an achievement degree #m to an achievement degree #m+1.

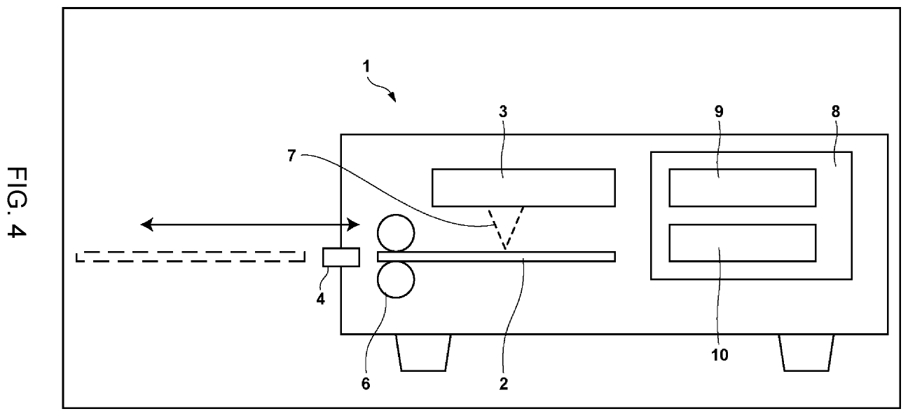

FIG. 4 is a cross-sectional view illustrating the measurement apparatus 1 according to an embodiment. Note that the reference sign 2 in FIG. 4 indicates a CI that has been conveyed to a measurement position of the measurement apparatus 1, and the dotted line indicates a CI before being inserted into the measurement apparatus 1. The CI that has been inserted into an insertion portion 4 of the measurement apparatus 1 is conveyed to the measurement position by a conveyance roller pair 6. A measurement unit3 measures the color of the discoloration area 12 of the CI. After the measurement of the CI, the CI is discharged outside the measurement apparatus 1 by rotating the conveyance roller pair 6 in a reverse direction. The measurement unit 3 reads an optical measurement value regarding the color value of the discoloration area 12 of the CI, e.g. spectral reflectance, using light 7. A control unit8 controls the measurement apparatus 1. Various types of information to be used in the measurement are stored in a storage unit 9 of the control unit 8. Also, a computational processing unit 10 of the control unit8 determines the color value of the discoloration area 12 of the CI based on a measurement result of the measurement unit 3, that is, spectral reflectance, for example. The control unit 8 outputs the color information indicating the determined color value to a PC 15.

Note that the measurement apparatus 1 shown in FIG. 4 discharges the CI from the insertion portion 4, but the configuration of the measurement apparatus 1 may be such that a discharging portion is provided on an opposite side of the insertion portion 4 relative to the measurement position of a CI, and the CI is discharged from the discharging portion after measurement. Also, the measurement apparatus 1 shown in FIG. 4 is configured to measure, after a CI has been conveyed to the measurement position, the CI by a fixed measurement unit 3, but the measurement apparatus 1 may be configured such that the measurement unit3 can be moved. That is, the configuration may also be such that the discoloration area 12 of a CI is read by moving the measurement unit 3 in a predetermined direction. Moreover, the configuration may also be such that a measurement apparatus 1 like a bar code reader is moved over a CI by an operator and reads the discoloration area 12 of the CI. Such a configuration is advantageous when CIs having various sizes are used.

The PC 15 determines the level of the sterilization process based on the color value of the discoloration area 12 acquired from the measurement apparatus 1, and transmits the determination result to the server apparatus 14. The server apparatus 14 records the received determination result to a database in association with a target identifier (herein after, target ID) for identifying the sterilization target or the set including the sterilization target and the sterilization processing date. Such information may constitute a part of the sterilization related information. Note that the database in which the sterilization related information is recorded may be provided in the server apparatus 14, or may be in an apparatus separated from the server apparatus 14.

One of the important objects of managing the sterilization related information is to appropriately utilize hospital resources by adjusting the frequency or timing of re-sterilization. In this specification, sterilizing a sterilization target used on a normal medical occasion is referred to as normal sterilization. The normal medical occasion may include an operation, a cure, and a treatment, for example, but there is no limitation thereto. In contrast, sterilizing again a sterilized sterilization target without being used on a normal medical occasion is referred to as re-sterilization. For example, according to the concept of the time-related sterility maintenance (TRSM), a medical instrument for which a predetermined storage period (e.g., several months after sterilization in a preferable storage state) determined by the manufacturer of the medical instrument or the hospital has expired is sent to a re-sterilization stage without being used. Also, a medical instrument that has been dropped in a work such as delivery or opening, or whose packaging is found to be defective is also sent to a re-sterilization stage without being used because there is a risk of bacteria being attached. Moreover, if it is determined that the sterilization process performed on a medical instrument is unsuccessful, as a result of verification performed at any point in time based on an indicator, the medical instrument is sent to a re-sterilization stage. However, in general, resources for sterilization (such as apparatuses for cleaning and sterilization, workspaces for assembly and packaging, human resources, and the like) that can be used in an individual hospital are limited. Therefore, performing re-sterilization on a medical instrument that origin ally need not be sterilized is not desirable because utilization of limited resources is hindered.

One of the approaches for optimizing the re-sterilization operation is to accumulate information regarding re-sterilization, analyze the tendency, specify a problem that induces the re-sterilization, and try to resolve the problem. For example, if it turns out from the statistical information that the frequency of re-sterilization with respect to a certain medical instrument is significant, the problem inherent to the sterilization workflow can be specified by checking the packaging, the storage place, the delivery work, and the opening work of the medical instrument. Also, if it turns out from the statistical information that many re-sterilization causes are originated from a certain storage place or a certain process stage, the occurrence frequency of re-sterilization can be reduced by improving the storage place or the process stage. As another approach, it is also possible to reduce the occurrence frequency of re-sterilization by introducing the concept of the event-related sterility maintenance (ERSM).

In view of these circumstances, in the present embodiment, every time the re-sterilization is performed, re-sterilization related information is recorded in a database, in the management system 100. The re-sterilization related information may be a part of the sterilization related information described above. Also, the management system 100 supports improvement in utilization of resources related to sterilization by generating statistical information regarding the re-sterilization based on the re-sterilization related information recorded in the past and presenting the statistical information to a user.

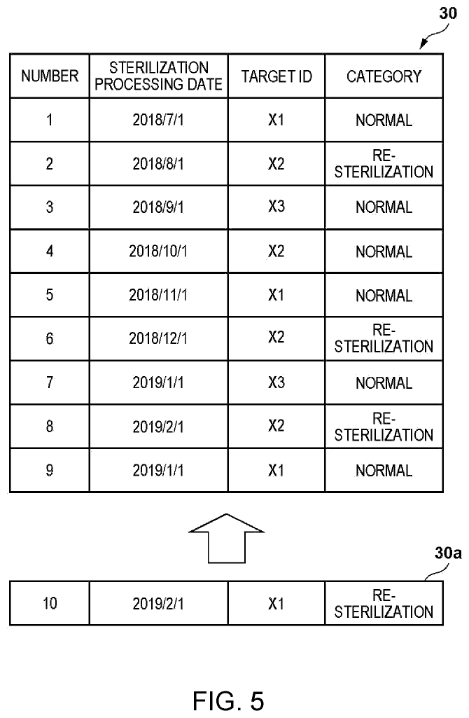

FIG. 5 shows a first example of a configuration of the sterilization related information to be recorded in a database in the management system 100. In the first example, the sterilization related information 30 includes four information items, namely a number, a sterilization processing date, a target ID, and a category. The “number” is a number for identifying an information record that is generated for every sterilization process. The “sterilization processing date” indicates a date on which the sterilization process has been performed on a set identified by the target ID. The “target ID” is, here, an identifier for uniquely identifying each set including at least one sterilization target. Note that the “target ID” may be an identifier for identifying each sterilization target instead of identifying each set. Also, the “target ID” may be an identifier for identifying a CI that is handled together with each set including at least one sterilization target, or an identifier for identifying a packaging member that is handled together with each set including at least one sterilization target. When the identifier for identifying a CI or a packaging member is replaced every time the sterilization process is performed, a separate unique identifier for management that is mapped to the identifiers may be introduced in order to identify the same set through a plurality of usages. The “category” is an information item (e.g., flag) indicating whether the corresponding sterilization process is a normal sterilization or a re-sterilization. For example, it is assumed that the sterilization process is again performed on a set identified by a target ID “X1” (hereinafter, set X1) on Feb. 1, 2019 without the set X1 having being used. In this case, the management system 100 records the “sterilization processing date” indicating Feb. 1, 2019 and the “category” information indicating that the sterilization process corresponds to a re-sterilization in the database in association with the “target ID”=“X1” as the re-sterilization related information. In FIG. 5, the record 30a corresponds to such re-sterilization related information.

Inputting the above-described sterilization related information 30 that may include the re-sterilization related information to the system may be performed at any point in time in the sterilization workflow. For example, a user that operates the sterilization processing apparatus 13 in the sterilization stage may in put the sterilization related information 30 to the system before starting the sterilization process, in the middle of the process, or after the process is ended. Alternatively, at a predetermined timing such as once per half day or once per day, the sterilization related information 30 regarding the sterilization processes that have been performed in a corresponding period may be input to the system. The category of the sterilization and another related information maybe transmitted from a user that has recognized the information to a user who makes an input via a PC 15 and the network of the system, or may be transmitted using a physical medium such as a paper form.

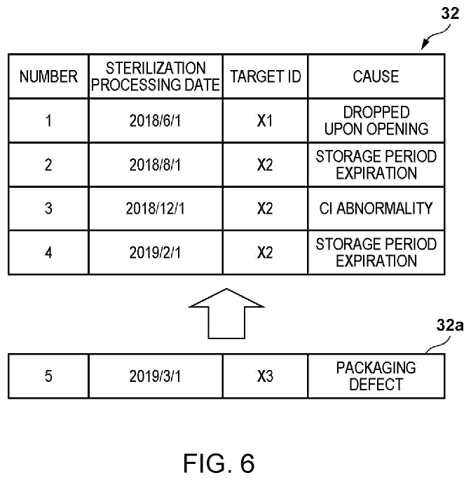

FIG. 6 shows a second example of the configuration of the sterilization related information to be recorded in the database, in the management system 100. In the second example, sterilization related information 32 only includes a record related to re-sterilization. The sterilization related information 32 includes four information items, namely a number, a sterilization processing date, a target ID, and a cause. The “cause” information is an information item indicating the cause of re-sterilization of the corresponding sterilization process. For example, it is assumed that the packaging of a set X3 is found to be damaged before using the set, and the sterilization process is again performed on the set X3 on Mar. 1, 2019. In this case, the management system 100 records the “sterilization processing date” information indicating Mar. 1, 2019, and the “cause” information indicating that the cause of re-sterilization corresponds to a “packaging defect” in the database in association with the “target ID”=“X3” as the re-sterilization related information. In FIG. 6, the record 32a corresponds to such re-sterilization related information. Note that the packaging defect may include various conditions such as a packaging damage, a sealing failure caused by an erroneous operation, a filter attachment failure, a scar caused by in sufficient durability, and filter degradation.

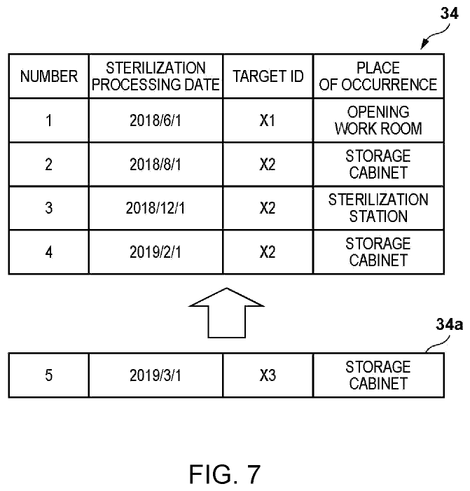

FIG. 7 shows a third example of the configuration of the sterilization related information to be recorded in the database, in the management system 100. In the third example, sterilization related information 34 only includes a record related to re-sterilization. The sterilization related information 34 includes four information items, namely a number, a sterilization processing date, a target ID, and a place of occurrence. The “place of occurrence” information is an information item indicating the place (including the place at which a cause was found) at which the cause of re-sterilization of the corresponding sterilization process occurred. For example, it is assumed that the cause of re-sterilization of a set X3 is a packaging damage when the set was delivered from the storage cabinet, and the sterilization process is again performed on the set X3 on Mar. 1, 2019. In this case, the management system 100 records the “sterilization processing date” information indicating Mar. 1, 2019, and the “place of occurrence” information indicating that the re-sterilization cause occurred at the “storage cabinet” in the database in association with the “target ID”=“X3” as the re-sterilization related information. In FIG. 7, the record 34a corresponds to such re-sterilization related information.

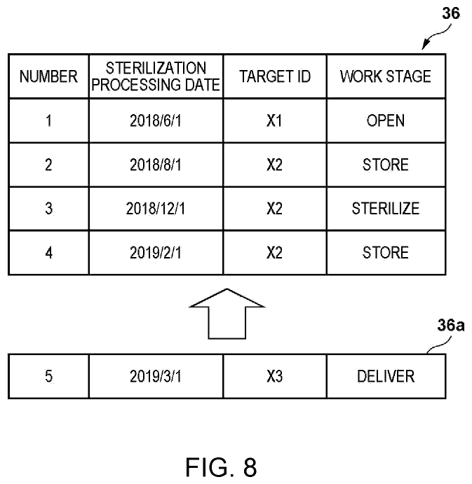

FIG. 8 shows a fourth example of the configuration of the sterilization related information to be recorded in the database, in the management system 100. In the fourth example, sterilization related information 36 only includes a record related to re-sterilization. The sterilization related information 36 includes four information items, namely a number, a sterilization processing date, a target ID, and a work stage. The “work stage” information is an information item indicating the work stage at which the cause of re-sterilization of the corresponding sterilization process occurred. The work stage here may include not only a typical work stage in the sterilization workflow shown in FIG. 1 but also another work stage such as stocktaking for quantity management. For example, it is assumed that the cause of re-sterilization of a set X3 occurs in delivery from the storage cabinet, and the sterilization process is again performed on the set X3 on Mar. 1, 2019. In this case, the management system 100 records the “sterilization processing date” information indicating Mar. 1, 2019, and the “work stage” information indicating that the cause of re-sterilization occurs in the “delivery” stage in the database in association with the “target ID”=“X3” as the re-sterilization related information. In FIG. 8, the record 36a corresponds to such re-sterilization related information.

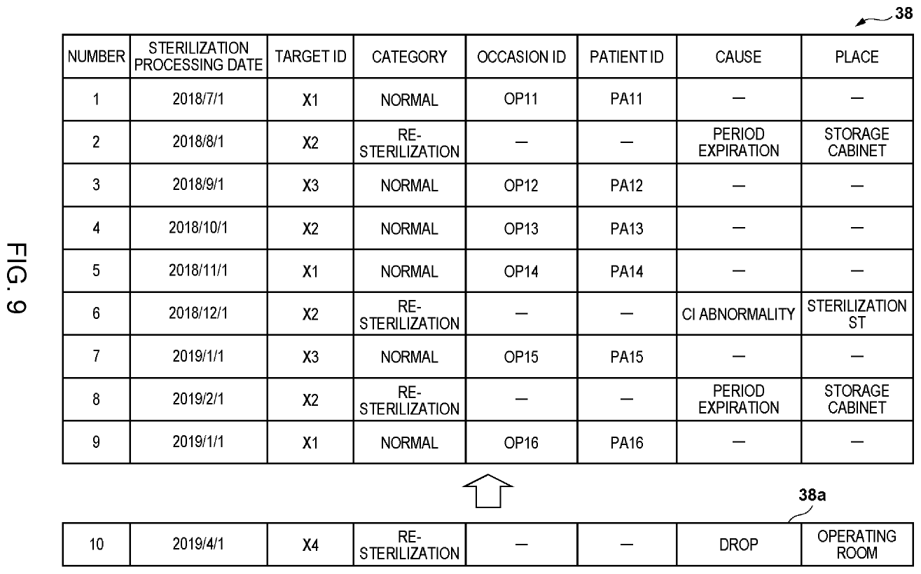

FIG. 9 shows a fifth example of the configuration of the sterilization related information to be recorded in the database, in the management system 100. In the fifth example, sterilization related information 38 includes eight information items, namely a number, a sterilization processing date, a target ID, a category, an occasion ID, a patient ID, a cause, and a place. In these information items, the “occasion ID” and the “patient ID” may include substantial contents only in records whose “category” is “normal”. In contrast, the “cause” and the “place” may include substantial contents only in records whose “category” is “re-sterilization”. The “occasion ID” is an identifier for uniquely identifying the medical occasion on which each sterilization target has been used. The medical opportunities may include an operation, a cure, and a treatment, for example, as described above. In a system in which only operations are targeted, an information item of “operation ID” may be defined instead of the “occasion ID”. The “patient ID” is an identifier for identifying the patient for which each sterilization target has been used. The “cause” is an information item indicating the cause information described using FIG. 6. The “place” is an information item indicating the place of occurrence information described using FIG. 7. For example, it is assumed that the sterilization process is again to be performed on a set X4 on Apr. 1, 2019, which is caused by an instrument belonging to the set X4 having been dropped in an operation room, without the set X4 having been used. In this case, the management system 100 records the “category” information indicating “re-sterilization”, the “cause” information indicating “dropped”, and the “place” information indicating an “operation room” in the database in association with the “target ID”=“X4” as the re-sterilization related information. In FIG. 9, the record 38a corresponds to such re-sterilization related information.

The sterilization related information or the re-sterilization related information that is to be recorded in the database by the management system 100 may include any combination of the information items described with reference to FIGS. 5 to 9. The information to be recorded may include an additional information item. Also, one or more information items described above may be omitted. For example, a “cause occurrence date” indicating the date on which the cause of re-sterilization has occurred may be recorded instead of (or in addition to) the “sterilization processing date” shown in FIGS. 5 to 9. Also, packaging member information (e.g., manufacturer name or model) indicating the type of the packaging member may be added as an information item. The information shown in one table format may be recorded in a plurality of tables in a distributed manner. Also, another recording format such as a tree format may be used instead of the table format.

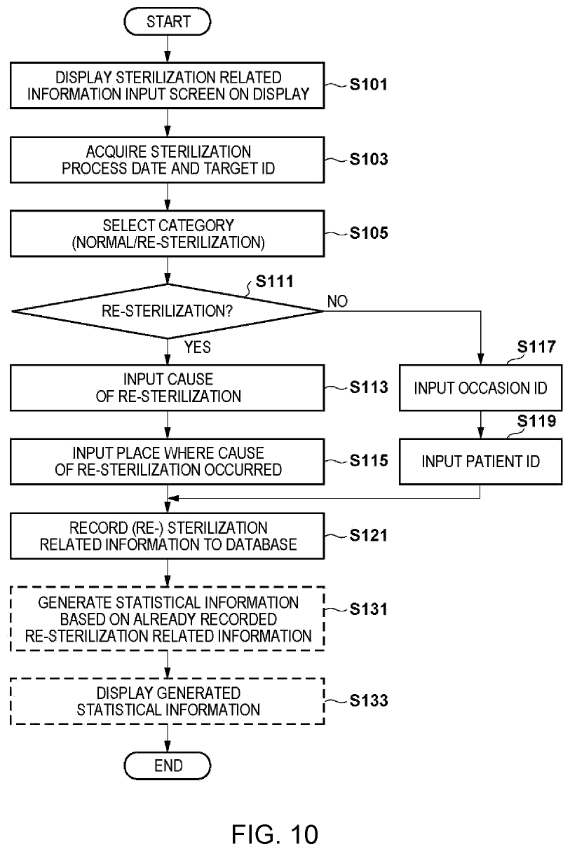

FIG. 10 is a flowchart illustrating an example of a processing flow for managing records with respect to sterilization targets on which the sterilization process is performed, according to the present embodiment. Here, it is assumed that the server apparatus 14 described using FIG. 2 records information acquired based on an input made by a user on a PC 15 in the database of the management system 100. The database is assumed to store sterilization related information as illustrated in FIG. 9. Note that the process step is abbreviated as S (step) in the following description.

First, in step S101, the server apparatus 14 displays an input screen for allowing a user to input sterilization related information in a display of a PC 15.

Next, in step S103, the server apparatus 14 acquires a sterilization processing date and a target ID for which sterilization related information should be input. The sterilization processing date and the target ID may be input or selected by the user on the input screen. Alternatively, in a case where the sterilization related information is input on a day on which the sterilization process is performed, the server apparatus 14 may automatically set the day as the sterilization processing date. Also, the server apparatus 14 may automatically acquire the target ID for identifying a set on which the sterilization process has been performed from the sterilization processing apparatus 13 via the network. When, a CI that is used along with a sterilization target set is provided with an identifier, the identifier that can be obtained through scanning performed by the measurement apparatus 1 may be automatically acquired as the target ID. Also, when a packaging member is provided with an identifier (e.g., bar code or IC tag), the identifier may be acquired by some reader apparatus.

Next, in step S105, the server apparatus 14 causes the user to select, on the input screen, the category of the sterilization process, that is, whether or not the sterilization process corresponds to re-sterilization. When the sterilization process performed on a set identified by the target ID acquired in step S103 corresponds to normal sterilization, “normal” is selected. On the other hand, when the sterilization process performed on the set corresponds to re-sterilization, “re-sterilization” is selected.

The processing thereafter branches depending on the category selected in step S105 (step S111). When the normal sterilization is selected, the processing is advanced to step S117. On the other hand, when the re-sterilization is selected, the processing is advanced to step S113.

When re-sterilization is selected as the category, in step S113, the server apparatus 14 causes the user to input the cause of the re-sterilization on the input screen. The causes of re-sterilization may be classified into several types in advance, and one of the types may be selected by the user. For example, the candidates of the cause of re-sterilization may include at least one of expiration of a predetermined storage period, a sterilization target having been dropped, a packaging defect, determination of unsuccessful sterilization based on the indicator. Also, an input field for allowing the user to freely input the cause that does not belong to any of the types may be provided on the input screen.

Next, in step S115, the server apparatus 14 causes the user to input, on the input screen, the place where the cause of re-sterilization has occurred. Some candidates of the place where the cause of re-sterilization has occurred may be defined in advance, and one of the candidates may be selected by the user. Also, although not illustrated in the diagram, the server apparatus 14 may further cause the user to input (select from a plurality of candidates, for example), on the input screen, the work stage at which the cause of re-sterilization has occurred.

Meanwhile, when normal sterilization has been selected as the category, in step S117, the server apparatus 14 causes the user to input the occasion ID on the input screen. The occasion ID uniquely identifies the occasion (operation or another medical occasion) in which a medical instrument belonging to the set identified by the target ID acquired in step S103 has been used. The occasion ID may be selected from occasion IDs of the latest medical opportunities registered in the database.

Next, in step S119, the server apparatus 14 causes the user to input the patient ID on the input screen. The patient ID uniquely identifies the patient for which a medical instrument belonging to the set identified by the target ID acquired in step S103 has been used. The patient ID may be selected form a list of patient IDs registered in the database, or may be automatically acquired by the server apparatus 14 based on the occasion ID acquired in step S117.

When inputting of information related to normal sterilization or re-sterilization is ended, in step S121, the server apparatus 14 records the sterilization related information (re-sterilization related information in the case of re-sterilization) to the database. These processes may be performed by a recording unit included in at least one of the server apparatus 14 and the PC 15. In this way, the management system 100 accumulates the sterilization related information including re-sterilization related information regarding the sterilization process that is repeatedly performed day by day in the database.

Thereafter, in step S131, which is indicated by dotted lines in the diagram, the server apparatus 14 generates statistical information regarding re-sterilization based on the re-sterilization related information recorded in the past. The statistical information may be generated in response to a request from a user accepted via the PC 15, or may be automatically generated in a cycle such as once a day or once a week. The statistical information may indicate the frequency of re-sterilization for each sterilization target or a ratio of a re-sterilization count relative the sterilization count. The frequency of re-sterilization is a re-sterilization count in a certain period, an average value of the re-sterilization count in a plurality of periods, or the like. The statistical information may be information in which the frequency of re-sterilization in each period is shown in time series with respect to a plurality of periods (the frequency of re-sterilization in one period may be simply referred to as a re-sterilization count). Additionally or alternatively, the statistical information may indicate the frequency of re-sterilization or the re-sterilization ratio described above for each cause of re-sterilization, for each place where the cause of re-sterilization has occurred, for each work stage in which the cause of re-sterilization has occurred, or for each type of the packaging member on which re-sterilization has been performed. This process may be performed by a generation unit included in at least one of the server apparatus 14 and the PC 15.

Next, in step S133, the server apparatus 14 causes the PC 15 to display a display screen for displaying the statistical information generated in step S131 in a display. This process may be performed by a presentation unit included in at least one of the server apparatus 14 and the PC 15. The user can analyze the tendency of re-sterilization or investigate the cause that triggered re-sterilization based on the displayed statistical information.

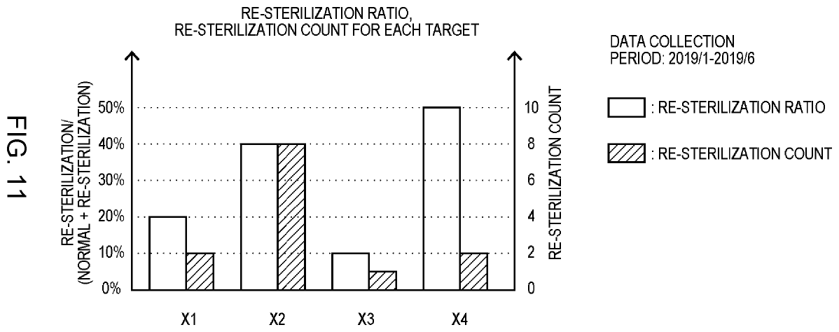

FIG. 11 shows a first example of the statistical information that may be generated based on the re-sterilization related information in the present embodiment. In the first example, the statistical information includes the re-sterilization ratio and the re-sterilization count for each sterilization target in a predetermined data collection period. The re-sterilization ratio may be derived by dividing the re-sterilization count in the period by all counts of the sterilization processes (that is, normal sterilization and re-sterilization). The user can easily analyze the tendency of re-sterilization by inspecting such statistical information. In the example in FIG. 11, the re-sterilization ratio and the re-sterilization count of a set X2 both show large values, and therefore it is meaningful to examine measures such as reviewing the storage method, the packaging method, or the storage period with respect to the set X2, for example. Also, the re-sterilization ratio of a set X4 shows a large value although the re-sterilization count is small, and therefore it may be meaningful to check whether or not a point to be improved is present with respect to the set X4. As in the example in FIG. 11, as a result of presenting the re-sterilization ratio in addition to the re-sterilization count as the statistical information, points to be improved regarding utilization of resources related to the sterilization can be easily specified.

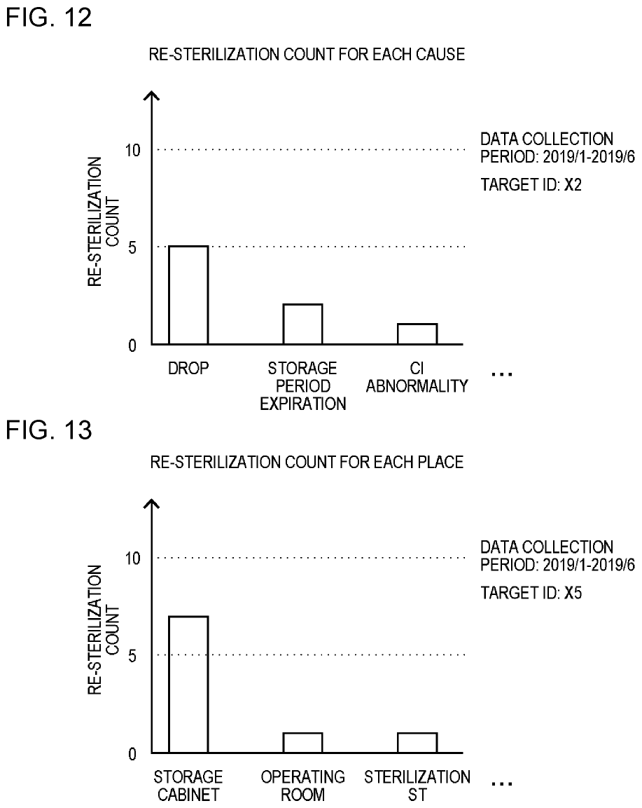

FIG. 12 shows a second example of the statistical information that may be generated based on the re-sterilization related information in the present embodiment. In the second example, the statistical information includes a re-sterilization count for each cause of re-sterilization in a predetermined data collection period with respect to a specific sterilization target. In the example in FIG. 12, the re-sterilization count of a set X2 for each cause of re-sterilization in a period from January to June in 2019 is shown as a graph in a descending order of the count. The user can immediately grasp the main cause of re-sterilization and examine measures by inspecting such statistical information with respect to a sterilization target for which the re-sterilization count is desired to be reduced. For example, in a case where a sterilization target having been dropped is a main cause, measures such as changing the storage place, replacement by another product whose shape is different, and alerting an operator can be considered. In a case where a defect such as a packaging damage is a main cause, measures such as also changing the storage place, replacement by a packaging material having higher strength, and alerting an operator can be considered.

FIG. 13 shows a third example of the statistical information that may be generated based on the re-sterilization related information in the present embodiment. In the third example, the statistical information includes a re-sterilization count for each place of occurrence of the cause of re-sterilization in a predetermined data collection period with respect to a specific sterilization target. In the example in FIG. 13, the re-sterilization count of a set X5 for each place of occurrence in a period from January to June in 2019 is shown as a graph in a descending order of the count. The user can immediately grasp the main place of occurrence of the cause of re-sterilization and examine measures by inspecting such statistical information with respect to a sterilization target for which there-sterilization count is desired to be reduced. For example, in a case where the cause of re-sterilization frequently occurs in a storage cabinet, a measure such as examining the configuration of the storage cabinet (shelf size or type, arrangement of sets, or the like) can be considered.

The statistical information of the present embodiment is not limited to the examples shown in FIGS. 11 to 13. For example, statistical information indicating the re-sterilization count for each work stage at which a cause of re-sterilization has occurred may be generated, for example. Also, for any of the parameters, the counting may be performed in any unit such as one specific sterilization target, a plurality of sterilization targets of the same type, or all sterilization targets. The display format of the statistical information is not limited to the illustrated bar graph format, and the format may be any format such as a pie chart, a scatter plot, or a table. Also, the statistical information may be presented to a user by being printed on a sheet or transmitted to another apparatus, instead of being displayed in a display.

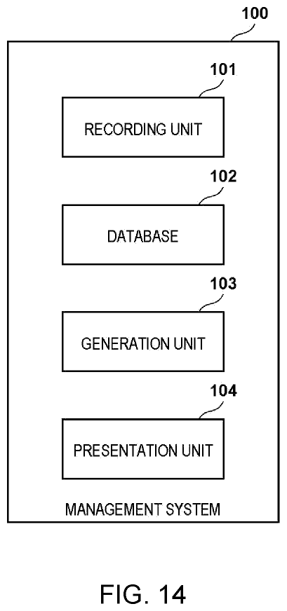

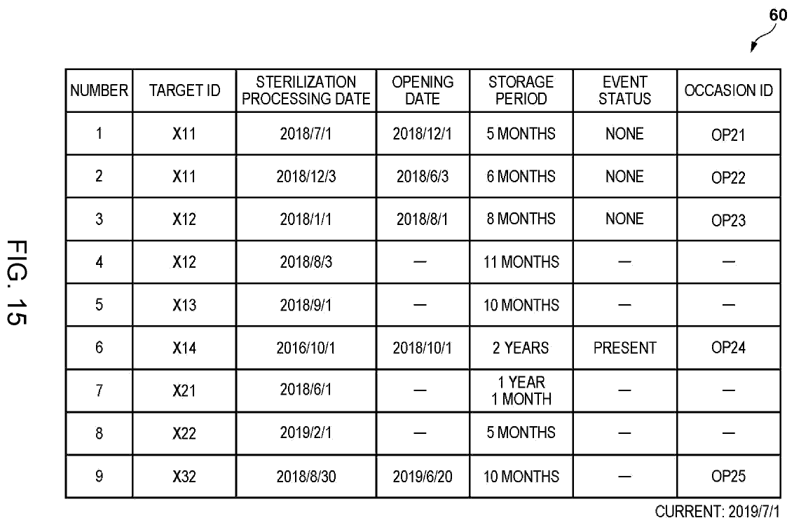

FIG. 14 is a block diagram illustrating an example of the functional configuration of the management system 100 according to the present embodiment. With reference to FIG. 14, the management system 100 includes a recording unit 101, a database 102, a generation unit 103, and a presentation unit 104.

The recording unit 101 may correspond to a processor of the server apparatus 14 or the PC 15 that writes data to the database 102. When a first sterilization process is again performed on a first sterilization target without the first sterilization target having been used, the recording unit 101 records the re-sterilization related information to the database in association with a target identifier for identifying the first sterilization target or a set including the first sterilization target, for example.

The database 102 includes a storage medium such as a hard disk or a semiconductor memory, and writes data to the storage medium and reads data from the storage medium in accordance with instructions from the server apparatus 14 or the PC 15. The database 102 may be a portion of the server apparatus 14, or may be present separately from the server apparatus 14. The database 102 stores the re-sterilization related information recorded by the recording unit 101.

The generation unit 103 may correspond to a processor of the server apparatus 14 or the PC 15 that reads out data from the database 102. The generation unit 103 generates statistical information related to re-sterilization based on the re-sterilization related information recorded in the past.

The presentation unit 104 may correspond to a processor of the server apparatus 14 or the PC 15 that can instruct to present information to a user. Alternatively, the presentation unit 104 may correspond to a display, a monitor, or a printer in the management system 100, or a communication interface of the server apparatus 14 or the PC 15. For example, the presentation unit 104 can present the statistical information generated by the generation unit 103 to a user by causing a display to display the statistical information. For another example, the presentation unit 104 may present the statistical information to the user by causing a printer to print the statistical information. Also, for another example, the presentation unit 104 may present the statistical information to the user by transmitting the statistical information to an apparatus that can be accessed by the user.

The recording unit 101, the generation unit 103, and the presentation unit 104 may also correspond to a user interface that can include at least one of a display, a keyboard, a mouse, a keypad, a touch panel, a printer, and a scanner of the server apparatus 14 or the PC 15. Such a user interface makes it possible for the user to operate the management system 100, input information to the management system 100, and inspect information output from the management system 100.

Note that the management system of the present embodiment includes the server apparatus 14 and at least one PC 15 that are connected by a network. That is, the management system 100 includes a plurality of computers connected via a network. However, the functions shown in FIG. 14 can be realized by one computer. That is, the management system of the present embodiment also includes a case of being realized by one computer in addition to the case of being realized by a plurality of computers. Also, in the case of being realized by one computer, this configuration may also be referred to as an information processing apparatus or a management apparatus instead of the management system. Moreover, the present embodiment can be realized by a program that, upon being executed by at least one computer, causes the at least one computer to function as the management system 100. This discussion is similarly applied to the other embodiments to be described in the following sections.

According to the present embodiment, statistical information based on re-sterilization related information recorded in the past is presented to a user. Therefore, the user can easily improve the method of utilizing limited resources for sterilization by analyzing the tendency of re-sterilization. With this, the safety or the productivity of medical activities is expected to be improved.

Also, according to the present embodiment, it may be shown whether or not each sterilization process corresponds to re-sterilization, in the sterilization related information that is recorded in the database in association with individual sterilization process. Therefore, it is possible to analyze in what target or in what condition re-sterilization has occurred at a high ratio (or at a low ratio).

Also, according to the present embodiment, the re-sterilization related information may include information indicating the cause of re-sterilization, the place where the cause of re-sterilization has occurred, the work stage at which the cause of re-sterilization has occurred, or the type of packaging member on which re-sterilization has performed. Therefore, the problem inherent to the sterilization workflow can be easily specified by performing statistical analysis with these factors being criteria.

Also, according to the present embodiment, a user selects whether or not the sterilization process corresponds to re-sterilization on an input screen for allowing the user to input the sterilization related information. Then, the input operation is switched depending on the selection between inputting information related to normal sterilization and inputting information related to re-sterilization. In this case, these input operations can be made at common timings, and the re-sterilization related information can be effectively collected. For example, a sterilization process having no corresponding occasion identifier or patient identifier is highly probable to correspond to re-sterilization. Therefore, it is also beneficial for collecting and accumulating re-sterilization related information that management system reminds the user of inputting re-sterilization related information with respect to such a sterilization process.

### Second Embodiment

The time-related sterility maintenance (TRSM) is a concept that the period in which sterility is effectively maintained after sterilization is specified by a time (period) based on conditions such as a packaging form of a sterilization target. In contrast, the event-related sterility maintenance (ERSM) is a concept that the period in which sterility is effectively maintained after sterilization is not a predetermined period, and is specified by a storage and transportation environment until the sterilization target is used. Previously, the TRSM has been the mainstream concept, but attempts for managing sterilization targets in accordance with the ERSM are increasing in recent years, with the technical advancement in packaging and storage and increasing awareness of the hospital efficient operation being the background. According to the ERSM, the number of times of performing re-sterilization due to expiration of a predetermined storage period even though the sterility is maintained can be reduced. Therefore, the appropriate operation of the ERSM promotes appropriate utilization of hospital resources regarding sterilization, and may contribute to improve the safety or the productivity of medical activities.

However, management examples in accordance with the ERSM are not sufficiently accumulated. Therefore, there is a need to prepare a mechanism for providing materials, to medical professionals, for determining whether a sterilization target that has been stored in a certain condition can be actually used on a medical occasion.

Therefore, in the present embodiment, the management system 100 manages records of sterilization target that are stored based on the ERSM. The records may be managed as a portion of the sterilization related information in the first embodiment or data in the same database, or may be managed separately from the sterilization related information in the first embodiment. The management system 100 presents materials for determining whether a sterilization target should be subjected to re-sterilization, whether a sterilization target can be used on a new medical occasion, or the like, based on storage records based on the ERSM.

Specifically, in the present embodiment, the management system 100 records a sterilization processing date on which the sterilization process has been performed on a set including at least one sterilization target to the database in association with an identifier (target ID) for identifying the set. Moreover, when a set is opened, the management system 100 records the opening date to the database. The storage period of a sterilization target (or its set) that was opened and used can be derived based on the recorded sterilization processing date and opening date. Also, when an event related to a certain sterilization target has occurred, the management system 100 may specify sterilization targets that should be subjected to re-sterilization from the other stored sterilization targets by searching the database based on the storage period of the sterilization target before the event has occurred.

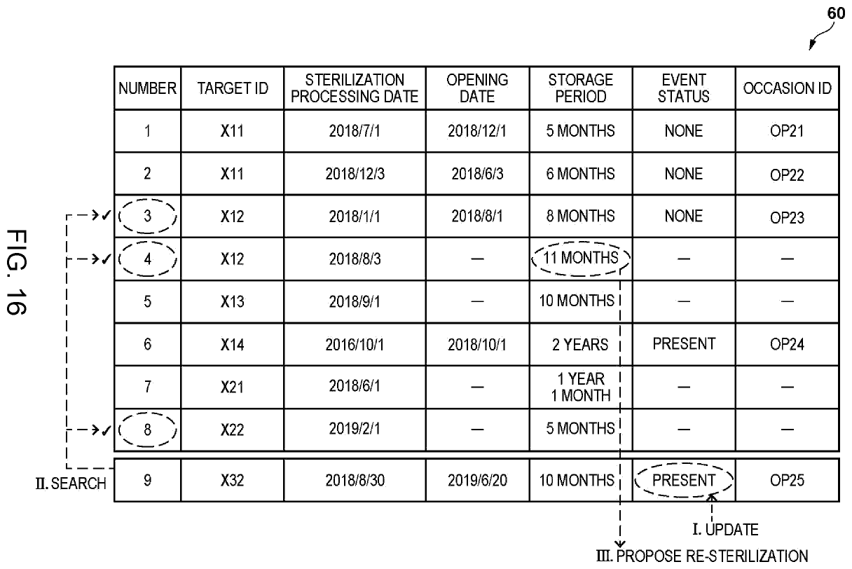

FIG. 15 shows an example of the configuration of ERSM management information to be recorded in the database of the management system 100 in the present embodiment. In the example in FIG. 15, the ERSM management information 60 includes seven information items, namely a number, a target ID, a sterilization processing date, an opening date, a storage period, an event status, and an occasion ID. The “number” is a number for identifying an information record that is generated every time one set of sterilization targets is stored. The “target ID” is an identifier for uniquely identifying each set including at least one sterilization target. The “sterilization processing date” indicates the date on which the sterilization process has performed on a set identified by the target ID. The “opening date” indicates the date on which a set identified by the target ID is opened. Of course, a date and time may be recorded in place of the date. The “opening date” field of a set whose storage has started but that is not yet opened may be empty. The “storage period” indicates the length of a period from the sterilization processing date until the opening date with respect to a set that has been opened, and the length of a period that has elapsed from the sterilization processing date until the current time with respect to a set that is not opened. The “event status” is information indicating whether or not a related event has occurred with respect to each set that has been opened and used in the past. The related event may include detection of bacteria by an irregular sampling survey, and occurrence of an infectious disease caused by the usage of a medical instrument, for example. A term such as an incident or an accident may be used instead of the event. If an occurrence of an event is not reported even if a predetermined period has elapsed since the set was opened or used, with respect to a set that was once opened and used, “None” may be set to the “event status” of the set. On the other hand, “Present” is set to the “event status” of a set with respect to which an occurrence of an event has been reported. The “event status” field of a set that has not been opened and a set with respect to which the predetermined period has not been elapsed from when the set was opened or used may be empty. The “occasion ID” is an identifier for uniquely identifying a medical occasion on which a sterilization target in a set identified by the target ID is used. The ERSM management information 60 may include a “patient ID” instead of the “occasion ID” (or in addition thereto) as the information item.

In the example in FIG. 15, the “opening date” field of each of sets X12, X13, X21, and X22 is empty, and these sets are being stored at the current time. For example, the storage period of the set X12 determined by a manufacturer or a hospital under the TRSM may be three months or six months, but the sterility of the set X12 may be maintained in a longer period in a preferable storage state due to a benefit of advancement in the packaging technique and the storage technique. Therefore, the hospital currently stores the set X12 in a period exceeding six months after sterilization in accordance with the ERSM, and the storage period reaches eleven months.

FIG. 16 is a diagram illustrating a proposal of re-sterilization based on the ERSM management information 60. It is assumed that, in a situation shown in the ERSM management information 60 in FIG. 15, as a result of a set X32 having been subjected to a sampling survey or used, an occurrence of an event is confirmed. The management system 100 updates the “event status” of the set X32 to “Present” (refer to I in FIG. 16). Next, the management system 100 searches the database based on the storage period of the set X32, which is related to the occurred event, before the occurrence of the event, and specifies a sterilization target(s) that should be subjected to re-sterilization, out of the other stored sterilization targets. In the example shown in FIG. 16, the storage period of the set X32 before the occurrence of the event is ten months, which corresponds to the gap between the sterilization processing date and the opening date in the ninth record. Here, sets X12 and X22 are assumed to be the same type as the set X32 (refer to II in FIG. 16). According to the third record, it can be understood that an event has not occurred with respect to the set X12 whose storage period is eight months. Also, when the fact that an event has occurred with respect to the set X32 whose storage period is ten months is also considered, it can be estimated that a safety risk may occur with respect to a set of the same type that is stored longer than eight months. The management system 100 recognizes that, as a result of the search, a set X12, out of the sets X12 to X22, is being stored in a period exceeding 10 months (refer to the fourth record). Therefore, the management system 100 specifies the set X12 that is being stored as a sterilization target that should be subjected to re-sterilization, and proposes re-sterilization of the set X12 to a user (refer to III in FIG. 16). The proposal of re-sterilization may be performed in any format such as a display of a message on a display or transmission of a message to a specific user terminal. Note that the extraction of a set of the same type may be performed based on a rule of the target ID, or based on comparison of an additional information item (e.g., “set type”), which is not illustrated, for example. Also, a set in which a packaging member of the same type is used may be treated as a re-sterilization target. For this purpose, the packaging member information indicating the type of the packaging member described above may be added to the ERSM management information 60.

The ERSM management information that is recorded to the database by the management system 100 may include an additional information item other than those shown in FIGS. 15 and 16. Also, one or more of the described information items may be omitted. For example, the ERSM management information may additionally include an information item indicating the achievement degree of each sterilization process. In this case, the condition for retrieving a sterilization target that should be subjected to re-sterilization may include the achievement degree of a corresponding sterilization process in addition to the storage period (a sterilization target whose achievement degree of the sterilization process is lower may be treated as the sterilization target that should be preferentially subjected to re-sterilization).

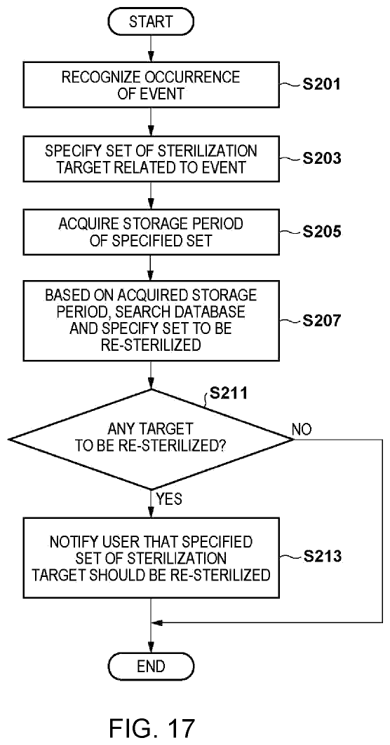

FIG. 17 is a flowchart illustrating a flow of processes that is performed in order to propose re-sterilization based on storage records of sterilization targets according to the present embodiment. Here, it is assumed that the server apparatus 14 described using FIG. 2 proposes re-sterilization, as required, based on the records accumulated in the database. The database is assumed to store the ERSM management information 60 as illustrated in FIGS. 15 and 16.

First, in step S201, the server apparatus 14 recognizes an occurrence of an event related to a stored sterilization target based on the ERSM. For example, as a result of a user inputting, to the management system 100, the target ID of a set including a sterilization target whose sterility is in doubt as a result of a sampling survey, an occurrence of an event related to the set may be recognized. For another example, as a result of the user inputting, to the management system 100, the occasion ID for identifying an operation that has caused an infectious disease, an occurrence of an event related to some sterilization target may be recognized. Also, for another example, as a result of the user in putting, to the management system 100, the patient ID of a patient who has been infected with an infectious disease, which has been caused by a specific medical occasion, an occurrence of an event related to a sterilization target that has been used for the patient may be recognized.

Next, in step S203, the server apparatus 14 specifies the set of a sterilization target related to the recognized event in the database. For example, the server apparatus 14 may specify a record of the sterilization target identified by the in put target ID in the ERSM management information 60. Also, the server apparatus 14 may determine which of the sterilization targets or the sets relates to the occurred event by collating the occasion ID or the patient ID that may be obtained in step S201 with occasion IDs or patient IDs that have already been recorded in the database.

Next, in step S205, the server apparatus 14 acquires the storage period, before the occurrence of the event, of the set specified in step S203 from the ERSM management information 60. The storage period acquired here is based on the sterilization processing date and the opening date that have been recorded with respect to the specified set.

Next, in step S207, the server apparatus 14 searches the database based on the storage period acquired in step S205, and specifies a set of a sterilization target that should be subjected to re-sterilization. For example, a sterilization target that belongs to the set whose type is the same as the set related to the occurred event and that is currently being stored in a period longer than a threshold value that is determined based on the storage period acquired in step S205 may be specified as the sterilization target that should be subjected to re-sterilization. Records of the past storage period that may be acquired in step S207 may be taken into consideration when the threshold value is set. For example, the longest storage period L₂ for which an event has not occurred with respect to sets of the same type may be set as the threshold value for determining re-sterilization. To prioritize safety, a value L₁-L₀ (or L₂-L₀) that is obtained by subtracting an offset L₀ from the storage period L₁ of the set with respect to which an event has occurred (or L₂) may also be set as the threshold value. Alternatively, a value αL₁ (or αL₂) obtained by multiplying the storage period L₁ (or L₂) by a coefficient a that is smaller than 1 may be set as the threshold value.

Next, in step S211, the server apparatus 14 determines whether at least one set that should be subjected to re-sterilization is specified in step S207. If no set is specified to be subjected to re-sterilization, the next step S213 is skipped, and the processing in FIG. 17 is ended.

If a set that should be subjected to re-sterilization is specified, in step S213, the server apparatus 14 notifies (proposes to or warns) the user that the set of the specified sterilization target should be subjected to re-sterilization. The user who has received this notification may arrange the re-sterilization as indicated by the notification, or may determine whether the re-sterilization is needed by performing further checking regarding the cause of the event.

The server apparatus 14 may not only execute the processing as shown in FIG. 17 upon recognizing an event, but also automatically execute the processing in a cycle such as once a day. When the processing is automatically executed, a set of the sterilization target whose storage period is determined to have reached a threshold value of the storage period that is dynamically set based on the past event occurrence situations may be specified by the server apparatus 14. Also, the server apparatus 14 may notify the user that the specified set should be subjected to re-sterilization. With this, the safety can be secured by performing re-sterilization on various sets at appropriate timings, without using a uniform storage period based on the TRSM.

In the present embodiment, the management system 100 may also have a function of presenting the event occurrence situation to a user in response to an inquiry from the user.

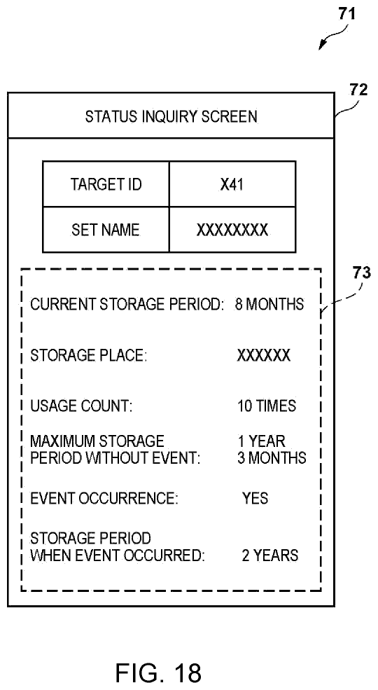

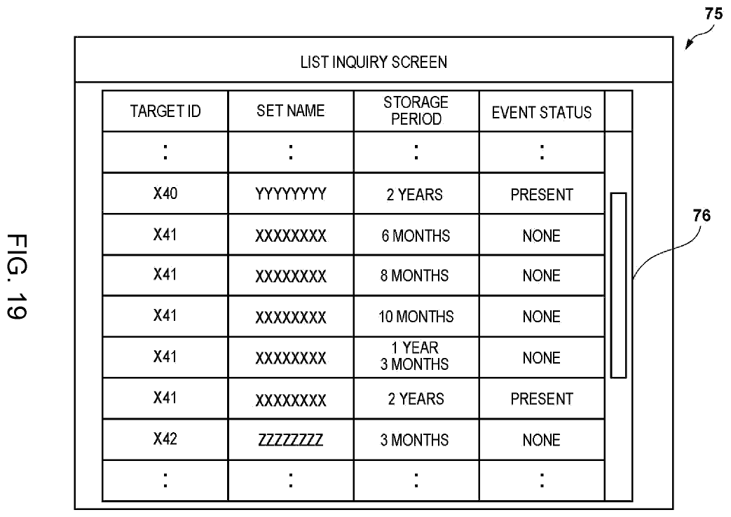

In a first example of presenting the event occurrence situation, the management system 100 may present the event occurrence situation with respect to at least one set, in response to an inquiry from the user, based on the event status of the set. FIG. 18 shows an example of a status inquiry screen in the first example. A status inquiry screen 71 shown in FIG. 18 includes an input field 72 and a presentation field 73. The input field 72 is a field for allowing a user to input an identifier or a name of the set with respect to which the user desires to inspect the event occurrence situation. The presentation field 73 is a field for presenting an event occurrence situation with respect to a set that is specified by the user input made to the input field 72. In the example in FIG. 18, a current storage period, a storage place, a usage count, a maximum storage period without an event, whether or not an event has occurred, and a storage period when an event has occurred are shown in the presentation field 73 as the event occurrence situation. The user can recognize that, by inspecting such an event occurrence situation, it is safe to continuously store a set X41 until a period of about one year and three months, and there may be a safety risk if the set X41 is continuously stored in a period of about two years, for example.

In a second example of presenting the event occurrence situation, the management system 100 may present the storage period and the event occurrence situation with respect to at least one set in a list format, in response to an inquiry from the user, based on the event status of the set. The management system 100 may present, when presenting in a list format, the storage periods respectively corresponding to a plurality of usages of one set, and corresponding event occurrence situations, in the order of storage period (ascending order or descending order). FIG. 19 shows an example of the list inquiry screen in the second example. The list inquiry screen 75 shown in FIG. 19 includes a presentation field 76. The presentation field 76 is a field for presenting the event occurrence situations, in a list format, with respect to a plurality of sets that are recorded in the database. In the example in FIG. 19, the presentation field 76 includes information items, namely a target ID, a set name, a storage period, and an event status, and the statuses of sets identified by same target IDs are shown in the ascending order of the storage period, downward in the list. The user can objectively examine how long sets of the sterilization targets can each be stored at the maximum under the ERSM, by inspecting such an event occurrence situation.

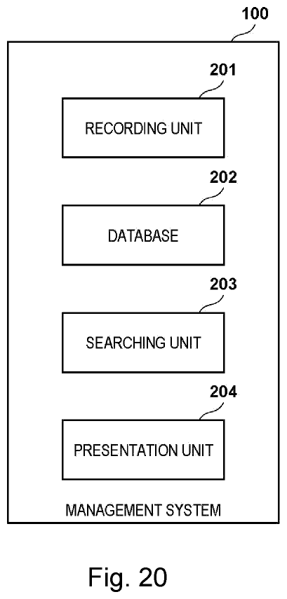

FIG. 20 is a block diagram illustrating an example of a functional configuration of the management system 100 according to the present embodiment. With reference to FIG. 20, the management system 100 includes a recording unit 201, a database 202, a searching unit 203, and a presentation unit 204.

The recording unit 201 may correspond to a processor of the server apparatus 14 or the PC 15 that writes data to the database 202. The recording unit 201 records a sterilization processing date on which the sterilization process has been performed and an opening date on which a set was opened to the database 202 with respect to at least one sterilization target, for example. The storage period may be derived based on these sterilization processing date and opening date. Also, the recording unit 201 further records an event status indicating whether or not a related event has occurred, to the database 202, with respect to each set of the sterilization target that was used in the past. The occasion ID or the patient ID may also be recorded in the database 202.

The database 202 includes a storage medium such as a hard disk or a semiconductor memory, and writes data to the storage medium and reads out data from the storage medium in accordance with an instruction from the server apparatus 14 or the PC 15. The database 202 may be a portion of the server apparatus 14, or may be present separately from the server apparatus 14. The database 202 stores information described above that is to be recorded by the recording unit 201.

The searching unit 203 may correspond to a processor of the server apparatus 14 or the PC 15 that executes searching in the database 202. For example, the searching unit 203 searches the database 202 based on the storage period of a first sterilization target, before the occurrence of the event, that relates to the occurred event to specify a sterilization target, out of the other stored sterilization targets, that should be subjected to re-sterilization, for example. The searching unit 203 may specify the sterilization target or the set that relates to the occurred event by collating an occasion identifier or a patient identifier that is acquired in association with the occurrence of an event with the records in the database 202.

The presentation unit 204 may correspond to a processor of the server apparatus 14 or the PC 15 that can instruct to present information to the user. Alternatively, the presentation unit 204 may correspond to a display, a monitor, or a printer in the management system 100, or a communication interface of the server apparatus 14 or the PC 15. For example, the presentation unit 204 may present, in response to an inquiry from the user, the event occurrence situation with respect to at least one set based on the event status of the set. For another example, the presentation unit 204 may present, in response to an inquiry from the user, the storage period and the event occurrence situation of at least one set in a list format based on the event status recorded in the database 202.

The recording unit 201, the searching unit 203, and the presentation unit 204 may also correspond to a user interface that may include at least one of a display, a keyboard, a mouse, a keypad, a touch panel, a printer, and a scanner of the server apparatus 14 or the PC 15. Such a user interface makes it possible for the user to operate the management system 100, to input information to the management system 100, and to inspect information output from the management system 100.

According to the present embodiment, when some event has occurred with respect to a sterilization target that is stored based on the ERSM, another stored sterilization target that should be subjected to re-sterilization can be specified through database search based on the storage period, before the occurrence of the event, of the sterilization target that relates to the event. Therefore, the necessity of a sterilization target being subjected to re-sterilization due to an expiration of a fixed storage period is reduced. Therefore, the frequency of re-sterilization can be reduced based on objective records. The records of sterilization targets that are recorded as the present embodiment may be utilized as an evidence for supporting applicability of the ERSM.

Also, according to the present embodiment, which of the sterilization targets or the sets relates to an occurred event may be specified through collation with the records in the database based on an occasion identifier or a patient identifier that is acquired in association with the occurrence of the event. Therefore, the sterilization target relating to the occurrence of an event can be reliably traced, and safer operation of sterilization based on the ERSM can be realized.

Also, according to the present embodiment, a status indicating whether or not a related event has occurred may be recorded in the database with respect to each set of the sterilization target that has been used in the past. Also, an event occurrence situation with respect to a specific set or various sets may be presented to the user based on these records. Therefore, medical professionals can easily determine whether an individual sterilization target can be actually used on a medical occasion, or to what degree a sterilization target can be stored before being subjected to re-sterilization.

[Modifications]

The present invention is not limited to the first and second embodiments described above, and various modification are possible. For example, the functions of the management system described in the first embodiment may be combined, in any combination, with the functions of the management system described in the second embodiment.

In an example of such a combination, the management system 100 may record, with respect to each set of the sterilization target, information indicating the length of a period in which the set has been stored to the database. Also, if a selection has been made that a first sterilization process with respect to a first sterilization target corresponds to re-sterilization, the management system 100 may specify a sterilization target, out of the other stored sterilization targets, that should be subjected to re-sterilization by searching the database based on the storage period of the first sterilization target. This search may be performed only if the cause of re-sterilization of the first sterilization process is a predetermined cause (e.g., indicator abnormality). According to such a combination, inputting of the sterilization related information as in the first embodiment and management of the storage period as in the second embodiment can be integrally handled. Therefore, the efficiency of management and operation of sterilization related information in a hospital can be improved.

In another modification, the mechanism of the first and second embodiments described above may be extended to a mechanism that extends across a plurality of medical institutions. For example, as a result of providing an event occurrence situation in a certain medical institution in response to an inquiry from another medical institution, a shortage of management examples in accordance with the ERSM can be supplemented, and the storage of the sterilization target over a more appropriate period can be realized.

[Other Embodiments]

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-131398, filed on Jul. 16, 2019, which is hereby incorporated by reference herein in its entirety.

1. A management system that manages records with respect to sterilization targets on which sterilization processes are to be performed, the management system comprising:
one or more processors; and
one or more memories that store computer instructions that, when executed by the one or more processors, cause the management system to perform operations including:
recording, when a first sterilization process is again performed on a first sterilization target without the first sterilization target having been used, re-sterilization related information to a database in association with a target identifier for identifying the first sterilization target or a set including the first sterilization target; and
generating statistical information regarding re-sterilization based on re-sterilization related information recorded in the past,
wherein the re-sterilization related information includes information indicating a cause of the re-sterilization.

2. The management system according to claim 1, wherein the operations further include recording sterilization related information related to an individual sterilization process to the database, and
wherein the re-sterilization related information is sterilization related information including information indicating that the first sterilization process corresponds to re-sterilization.

3. The management system according to claim 1, wherein the cause of the re-sterilization is selected from candidates including at least one of an expiration of a predetermined storage period, the first sterilization target being dropped, a packaging defect, and a determination that sterilization is unsuccessful based on an indicator.

4. The management system according to claim 1, wherein the re-sterilization related information further includes a place or a work stage at which a cause of the re-sterilization has occurred, or information indicating a type of a packaging member to be subjected to re-sterilization.

5. The management system according to claim 2, wherein the operations include causing a display of a terminal apparatus to display an input screen for allowing a user to input the sterilization related information, and allowing the user to select on the input screen whether or not the sterilization process corresponds to re-sterilization.

6. The management system according to claim 5, wherein, if a selection has been made that the sterilization process corresponds to re-sterilization, the operations further include allowing the user to input a cause of the re-sterilization.

7. The management system according to claim 5, wherein, if a selection has been made that the sterilization process corresponds to re-sterilization, the operations further include allowing the user to input a place or a work stage at which a cause of the re-sterilization has occurred.

8. The management system according to claim 6, wherein, if a selection has been made that the sterilization process does not correspond to re-sterilization, the operations further include allowing the user to input an occasion identifier for identifying an occasion on which the first sterilization target has been used or a patient identifier for identifying a patient.

9. The management system according to claim 1, wherein the statistical information indicates, for each sterilization target, a frequency of re-sterilization or a ratio of a re-sterilization count relative to a sterilization count.

10. The management system according to claim 1, wherein the statistical information indicates a frequency of re-sterilization or a ratio of a re-sterilization count relative to a sterilization count for each cause of re-sterilization, for each place at which a cause of re-sterilization has occurred, for each work stage at which a cause of re-sterilization has occurred, or for each type of packaging member subjected to re-sterilization.

11. The management system according to claim 1, wherein the operations further include presenting, to a user, the statistical information related to re-sterilization that is generated based on the re-sterilization related information.

12. The management system according to claim 1, wherein the operations further include recording, for each set of a sterilization target, information indicating a storage period of the set to the database, and
wherein the management system, if a selection has been made that the first sterilization process with respect to the first sterilization target corresponds to re-sterilization, specifies a sterilization target, out of other stored sterilization targets, that should be subjected to re-sterilization by searching the database based on a storage period of the first sterilization target.

13. An information processing apparatus that manages records with respect to sterilization targets on which sterilization processes are to be performed, the information processing apparatus comprising:
one or more processors; and
one or more memories that store computer instructions that, when executed by the one or more processors, cause the information processing apparatus to perform operations including:
recording, when a first sterilization process is again performed on a first sterilization target without the first sterilization target having been used, re-sterilization related information to a database in association with a target identifier for identifying the first sterilization target or a set including the first sterilization target; and
generating statistical information regarding re-sterilization based on re-sterilization related information recorded in the past,
wherein the re-sterilization related information includes information indicating a cause of the re-sterilization.

14. A computer-readable storage medium storing a program, wherein the program causes a computer of the information processing apparatus according to claim 13 to perform the operations.

15. A management method in a management system that manages records with respect to sterilization targets on which sterilization processes are to be performed, the management method comprising:
recording, when a first sterilization process is again performed on a first sterilization target without the first sterilization target having been used, re-sterilization related information to a database in association with a target identifier for identifying the first sterilization target or a set including the first sterilization target; and
generating statistical information regarding re-sterilization based on re-sterilization related information recorded in the past,
wherein the re-sterilization related information includes information indicating a cause of the re-sterilization.

16. A management system that manages records with respect to sterilization targets that are stored based on event-related sterility maintenance, the management system comprising:
one or more processors; and
one or more memories that store computer instructions that, when executed by the one or more processors, cause the management system to perform operations including:
recording a sterilization processing date on which a sterilization process has been performed with respect to at least one set of a sterilization target to a database; and
searching the database based on a storage period, before an occurrence of an event, for a first sterilization target that relates to the occurred event to specify a sterilization target, out of other stored sterilization targets, that should be subjected to re-sterilization.

17. The management system according to claim 16, wherein the operations further include recording an opening date on which the at least one set was opened in the database, and
wherein the storage period of the first sterilization target is based on the sterilization processing date and the opening date that have been recorded with respect to the first sterilization target.

18. The management system according to claim 16, wherein the operations further include:
recording, to the database, an occasion identifier for identifying an occasion or a patient identifier for identifying a patient for which a sterilization target in the at least one set has been used, and
specifying a sterilization target or a set that relates to an occurred event by collating an occasion identifier or a patient identifier that is acquired in association with the occurred event with a recorded occasion identifier or a recorded patient identifier in the database.

19. The management system according to claim 16, wherein the operations further include recording a status indicating whether or not a related event has occurred to the database for each set of a sterilization target that has been used in the past.

20. The management system according to claim 19, wherein the operations further include presenting an event occurrence situation with respect to at least one set based on the status of the set in response to an inquiry from a user.

21. The management system according to claim 19, wherein the operations further include presenting a storage period and an event occurrence situation with respect to at least one set based on the status of the set in a list format in response to an inquiry from a user.

22. The management system according to claim 21, wherein the operations include presenting storage periods respectively corresponding to a plurality of usages of one set and corresponding event occurrence situations in the order of the storage period.

23. An information processing apparatus that manages records with respect to sterilization targets that are stored based on event-related sterility maintenance, the information processing apparatus comprising:
one or more processors; and
one or more memories that store computer instructions that, when executed by the one or more processors, cause the information processing apparatus to perform operations including:
recording a sterilization processing date on which a sterilization process has been performed with respect to at least one set of a sterilization target to a database; and
searching the database based on a storage period, before an occurrence of an event, for a first sterilization target that relates to the occurred event to specify a sterilization target, out of other stored sterilization targets, that should be subjected to re-sterilization.

24. A computer-readable storage medium storing a program, wherein the program causes a computer of the information processing apparatus according to claim 23 to perform the operations.

25. A management method in a management system that manages records with respect to sterilization targets that are stored based on event-related sterility maintenance, the management method comprising:
recording to a database a sterilization processing date on which a sterilization process has been performed with respect to at least one set of a sterilization target; and
searching the database based on a storage period, before an occurrence of an event, for a first sterilization target that relates to the occurred event to specify a sterilization target, out of other stored sterilization targets, that should be subjected to re-sterilization.
